(19) United States
(12) Patent Application Publication
(10) Pub. No.: US 2023/0034444 A1
(43) Pub. Date: Feb. 2, 2023

(54) SYSTEM FOR PREDICTING ADVERSE EVENT IN PATIENT

(71) Applicants: THE ASAN FOUNDATION, Seoul (KR); UNIVERSITY OF ULSAN FOUNDATION FOR INDUSTRY COOPERATION, Ulsan (KR)

(72) Inventors: Chang-Min CHOI, Seoul (KR); Jae-Cheol LEE, Seoul (KR)

(21) Appl. No.: 17/792,454

(22) PCT Filed: Feb. 2, 2021
(86) PCT No.: PCT/KR2021/001327

§ 371 Date: Jul. 13, 2022

(30) Foreign Application Priority Data
Mar. 26, 2020 (KR) 10-2020-0037071

(51) Int. Cl. A61B 5/00; A61B 5/0205; G16H 80/00; G16H 40/67

(52) U.S. Cl. CPC A61B 5/7275; A61B 5/0205; A61B 5/7246; G16H 80/00; G16H 40/67; A61B 5/024

(57) ABSTRACT

A system for predicting an adverse reaction of a patient, based on a real-time bio-signal of the patient, according to an embodiment of the disclosure, may include: a bio-signal measurement device configured to measure a bio-signal of the patient at a first time interval and transmit a measured result to a patient terminal at the first time interval; the patient terminal configured to collect the bio-signal measured at the first time interval from the bio-signal measurement device and transmit the collected bio-signal to a server at a second time interval; the server configured to receive the bio-signal from the patient terminal at the second time interval, based on the received bio-signal, identify whether a predetermined action is necessary for the patient at the second time interval, and when the predetermined action is necessary, transmit a message to a medical team terminal; and the medical team terminal configured to receive the message from the server when the predetermined action is necessary.

### TECHNICAL FIELD

The disclosure relates to a patient adverse reaction prediction system, and more particularly, to a system for predicting an adverse reaction of a patient, based on a real-time bio-signal of the patient and taking action accordingly.

### BACKGROUND ART

Recently, as the social environment has rapidly changed, interest in health is increasing and health care costs are also increasing. In addition, with the rapid development of IT technology, IT technology is applied to the medical field, and research and development in the healthcare field based on the network environment is budding.

However, despite the increase in healthcare expenditure and the development of new medical services, when adverse reactions occur in the daily lives of patients, abnormal symptoms are not detected in advance, and thus, it is often the case that appropriate medical services are not provided.

In addition, due to an increase in the rapid-aging elderly population in society, an increase in dual-income households, an increase in physical distance between families due to the nuclearization of family, a rapid increase in facilities where the elderly concentrate, such as postpartum care centers and senior homes, and lack of medical care due to a decrease in rural population, the number of cases where appropriate actions may not be taken when an adverse reaction occurs in a patient is also increasing.

As such, according to the aging and the nuclearization of family, when an adverse reaction occurs in a patient, early detection and prompt first aid are social issues.

Also, a rate of sudden death of the general public due to excessive work and stress is also increasing, and accordingly, the need for a system or device capable of checking the health status of each individual by an expert, even in a place which is not a hospital, is increasing.

### DESCRIPTION OF EMBODIMENTS

### Technical Problem

The disclosure is directed to predicting adverse events in patients who are active in places other than hospitals or out of reach of a medical team, and taking appropriate action accordingly.

Also, the disclosure is directed to detecting an adverse event in a patient in real time by monitoring a bio-signal of the patient in real time, and more accurately detecting an adverse event considering the special needs of individual patients.

As such, the disclosure is directed to identifying a real-time state of a patient from a remote place by reducing a time difference between an actual state of the patient and a state identified by a medical team when an adverse event occurs to a patient, and to more effectively utilizing resources of a bio-signal measurement device and a patient terminal by transmitting a bio-signal according to a relatively long time interval when an adverse event does not occur.

Also, the disclosure is directed to enabling miniaturization of a bio-signal measurement device by omitting a relatively bulky large battery, and accordingly, to increasing the ease of attachment of the bio-signal measurement device to the body of a patient.

Also, the disclosure is directed to reducing manufacturing cost of a bio-signal measurement device by allowing the bio-signal measurement device to transmit a bio-signal to a server via a patient terminal that is always connected to a communication network.

### Solution to Problem

A system for predicting an adverse reaction of a patient, based on a real-time bio-signal of the patient, according to an embodiment of the disclosure, may include: a bio-signal measurement device configured to measure a bio-signal of the patient at a first time interval and transmit a measured result to a patient terminal at the first time interval; the patient terminal configured to collect the bio-signal measured at the first time interval from the bio-signal measurement device and transmit the collected bio-signal to a server at a second time interval; the server configured to receive the bio-signal from the patient terminal at the second time interval, based on the received bio-signal, identify whether a predetermined action is necessary for the patient at the second time interval, and when the predetermined action is necessary, transmit a message to a medical team terminal; and the medical team terminal configured to receive the message from the server when the predetermined action is necessary.

The bio-signal of the patient may include at least one of a saturation level of oxygen in the body of the patient and a heart rate of the patient.

The server may be configured to receive the bio-signal of the patient from the patient terminal at a first time point, and when at least one pattern of an oxygen saturation level pattern and a heart rate pattern within a predetermined time period from the first time point corresponds to a pre-stored adverse reaction pattern, determine that the predetermined action is necessary for the patient, and transmit a message including at least one of the oxygen saturation level pattern and the heart rate pattern to the medical team terminal.

The server may be configured to, when it is determined that the predetermined action is necessary for the patient, request the patient terminal to transmit the bio-signal to the server at a second-first time interval that is shorter than the second time interval, request that the bio-signal measurement device measure the bio-signal of the patient at a first-first time interval that is shorter than the first time interval via the patient terminal and transmit the measured bio-signal to the patient terminal, and receive the bio-signal from the patient terminal at the second-first time interval and identify whether an additional action is necessary for the patient at the second-first time interval.

The server may be configured to, when the predetermined action is necessary for the patient, collect the bio-signal received at the second-first time interval and transmit the message to the medical team terminal at a third time interval.

The bio-signal measurement device and the patient terminal may be connected via a first communication scheme, the patient terminal and the medical team terminal may be connected to a communication network via a second communication scheme, and the server may be connected to the communication network via a third communication scheme.

When the bio-signal measurement device is not capable of transmitting the bio-signal to the patient terminal via the first communication scheme, the bio-signal measurement device may be connected to the communication network via a fourth communication scheme, and then may transmit the bio-signal to the patient terminal via the communication network.

The patient terminal may transmit the bio-signal received from the bio-signal measurement device via the communication network to the server via the communication network.

The first communication scheme and the fourth communication scheme may be one of a Bluetooth scheme, a worldwide interoperability for microwave access (WiMAX) scheme, a wireless fidelity (Wi-Fi) scheme, a ZigBee scheme, a wireless body area network (WBAN) scheme, and a medical body area network (MBAN) scheme.

The second communication scheme may be one of a long-term evolution (LTE) scheme, an LTE advanced scheme, an evolution-data optimized (EV-DO) scheme, a wireless broadband (Wibro) scheme, a high speed downlink packet access (HSPDA) scheme, a code division multiplex access 2000 (CDMA2000) scheme, a global system for mobile communication (GSM) scheme, and a wideband code division multiplex access (WCDMA) scheme.

The third communication scheme may be one of a digital subscriber line (DSL) scheme, an x digital subscriber line (xDSL) scheme, a hybrid fiber coaxial (HFC) scheme, a fiber to the office (FTTO) scheme, a fiber to the curb (FTTC) scheme, a fiber to the home (FTTH) scheme, a local multipoint distribution service (LMDS) scheme, and a multichannel multipoint distribution service (MMDS) scheme.

The communication network may be an Internet network.

When the patient terminal fails to receive the bio-signal from the bio-signal measurement device at the first time interval or fails to transmit the collected bio-signal to the server at the second time interval, the patient terminal may generate an alarm in a preset manner.

### Advantageous Effects of Disclosure

According to the disclosure, adverse events in patients who are active in places other than hospitals or out of reach of a medical team can be predicted, and appropriate action can be taken accordingly.

Also, an adverse event in a patient can be detected in real time by monitoring a bio-signal of the patient in real time, and at this time, an adverse event can be more accurately detected considering the specificity of individual patients.

Also, when an adverse event occurs to a patient, a real-time state of the patient can be identified from a remote place by reducing a time difference between an actual state of the patient and a state identified by a medical team, and when an adverse event does not occur, resources of a bio-signal measurement device and a patient terminal can be more effectively utilized by transmitting a bio-signal according to a relatively long time interval.

Also, a bio-signal measurement device can be miniaturized by omitting a relatively bulky large battery, and accordingly, the ease of attachment of the bio-signal measurement device to the body of a patient increases.

Also, manufacturing cost of a bio-signal measurement device can be reduced by allowing the bio-signal measurement device to transmit a bio-signal to a server via a patient terminal that is always connected to a communication network.

### BRIEF DESCRIPTION OF DRAWINGS

FIG. 1 is a diagram showing a configuration example of a patient adverse reaction prediction system according to an embodiment of the disclosure.

FIG. 2 is a diagram for explaining a configuration of a bio-signal measurement device configured to measure a saturation level of oxygen in the body and a heart rate, according to an embodiment of the disclosure.

FIG. 3 is a block diagram for explaining internal configurations of a patient terminal, a medical team terminal, and a server, according to an embodiment of the disclosure.

FIG. 4 is a diagram for explaining a communication scheme between each element of an adverse reaction prediction system, according to an embodiment of the disclosure.

FIG. 5 is an example of a measurement result screen displayed on a patient terminal, according to an embodiment of the disclosure.

FIG. 6 is an example of a monitoring screen for a specific patient, which is displayed on a server, according to an embodiment of the disclosure.

FIG. 7 is an example of a message reception screen displayed on a medical team terminal in the event of an adverse event in a specific patient.

FIG. 8 is a flowchart for explaining a method, performed by a patient adverse reaction prediction system, of identifying a bio-signal of a patient in real time, and a method of predicting an adverse event in a patient based thereon.

### BEST MODE

A system for predicting an adverse reaction of a patient, based on a real-time bio-signal of the patient, according to an embodiment of the disclosure, may include: a bio-signal measurement device configured to measure a bio-signal of the patient at a first time interval and transmit a measured result to a patient terminal at the first time interval; the patient terminal configured to collect the bio-signal measured at the first time interval from the bio-signal measurement device and transmit the collected bio-signal to a server at a second time interval; the server configured to receive the bio-signal from the patient terminal at the second time interval, based on the received bio-signal, identify whether a predetermined action is necessary for the patient at the second time interval, and when the predetermined action is necessary, transmit a message to a medical team terminal; and the medical team terminal configured to receive the message from the server when the predetermined action is necessary.

### Mode of Disclosure

The detailed description of the disclosure to be described below refers to the accompanying drawings, in which specific embodiments by which the disclosure may be implemented are shown. These embodiments are described in sufficient detail to enable those of ordinary skill in the art to implement the disclosure. It should be understood that the various embodiments of the disclosure are different but need not be mutually exclusive. For example, certain shapes, structures, and characteristics described herein may be implemented with changes from one embodiment to another without departing from the spirit and scope of the disclosure. In addition, it should be understood that the location and arrangement of individual components in each embodiment may be changed without departing from the sprit and the scope of the disclosure. Therefore, the following detailed description should not be limited by the description, and the scope of the disclosure should be taken as encompassing the scope of the claims and all equivalents thereto. In the drawings, like reference numerals refer to the same or similar components throughout the various aspects.

Hereinafter, in order to enable those of ordinary skill in the art to easily implement the disclosure, various embodiments of the disclosure will be described in detail with reference to the accompanying drawings.

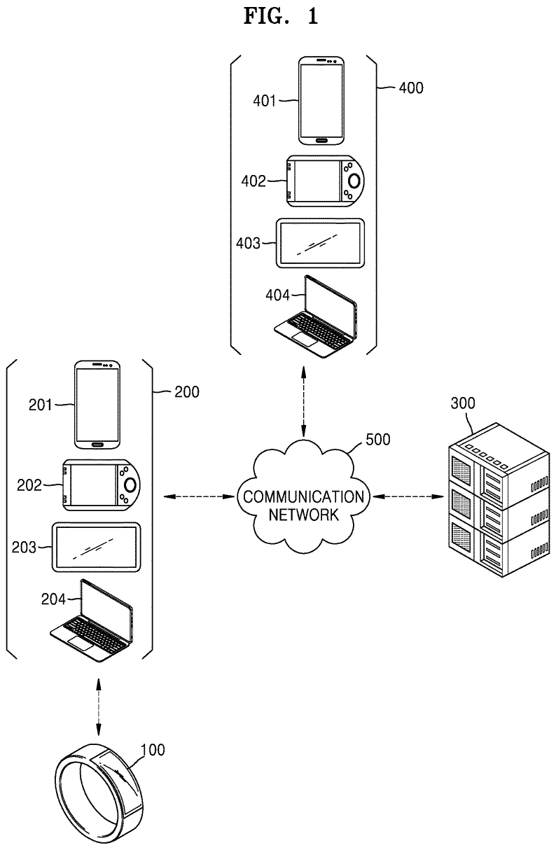

FIG. 1 is a diagram showing a configuration example of a patient adverse reaction prediction system according to an embodiment of the disclosure.

The system of FIG. 1 shows an example including a bio-signal measurement device 100 for measuring a bio-signal of a patient, a plurality of patient terminals 201, 202, 203, and 204, a server 300, a plurality of medical team terminals 401, 402, 403, and 404, and a communication network 500. FIG. 1 is an example for explaining the disclosure, and the forms of the devices or the number of device is not limited to the ones in FIG. 1.

The bio-signal measurement device 100 may refer to various devices capable of measuring a bio-signal of a patient and transmitting the bio-signal to the plurality of patient terminals 201, 202, 203, and 204. For example, the bio-signal measurement device 100 may be a device that measures at least one of a saturation level of oxygen in the body of a patient and a heart rate of the patient in real time and transmits the at least one of a saturation level of oxygen in the body of a patient and a heart rate of the patient to the plurality of patient terminals 201, 202, 203, and 204. In this regard, the saturation level of oxygen in the body and the heart rate are examples of measurement items that the bio-signal measurement device 100 may measure, and the spirit of the disclosure is not limited thereto.

The bio-signal measurement device 100 may invasively or non-invasively measure a bio-signal of a patient. For example, the bio-signal measurement device 100 may non-invasively measure a saturation level of oxygen in the body and a heart rate of a patient. Also, the bio-signal measurement device 100 may invasively measure a blood glucose level, a hemoglobin level, or the like.

The bio-signal measurement device 100 may be attached to the body of a patient to measure a bio-signal of the patient in real time and may transmit the bio-signal to the plurality of patient terminals 201, 202, 203, and 204. The bio-signal transmitted to the plurality of patient terminals 201, 202, 203, and 204 may be transmitted to the server 300 and used for the server 300 to determine whether a predetermined action is necessary for the patient. A detailed description thereof is provided below.

The plurality of patient terminals 201, 202, 203, and 204 (hereinafter, referred to as a patient terminal 200) and the plurality of medical team terminals 401, 402, 403, and 404 (hereinafter, referred to as a medical team terminal 400) may be a stationary terminal implemented as a computer device or a mobile terminal.

Examples of the patient terminal 200 and the medical team terminal 400 may include a smartphone, a portable phone, a navigation device, a computer, a laptop, a digital broadcasting terminal, a personal digital assistant (PDA), a portable multimedia player (PMP), and a tablet personal computer (PC).

The patient terminal 200 and the medical team terminal 400 may communicate with the patient terminal 200, the medical team terminal 400, and/or the server 300 via the communication network 500 by using a wireless or wired communication scheme.

For example, when the patient terminal 200 and the medical team terminal 400 are respectively smartphones 201 and 401, the patient terminal 200 and the medical team terminal 400 may be connected to the communication network 500 according to a wireless communication scheme, such as a long-term evolution (LTE) scheme, an LTE advanced scheme, an evolution-data optimized (EV-DO) scheme, a wireless broadband (Wibro) scheme, a high speed downlink packet access (HSPDA) scheme, a code division multiplex access 2000 (CDMA2000) scheme, a global system for mobile communication (GSM) scheme, and a wideband code division multiplex access (WCDMA) scheme.

When the patient terminal 200 and the medical team terminal 400 are respectively computers 204 and 404, the patient terminal 200 and the medical team terminal 400 may be connected to the communication network 500 according to a wired communication scheme, such as a digital subscriber line (DSL) scheme, an x digital subscriber line (xDSL) scheme, a hybrid fiber coaxial (HFC) scheme, a fiber to the office (FTTO) scheme, a fiber to the curb (FTTC) scheme, a fiber to the home (FTTH) scheme, a local multipoint distribution service (LMDS) scheme, and a multichannel multipoint distribution service (MMDS) scheme.

In addition, in the disclosure, the communication network 500 may refer to one huge computer communication network, such as an Internet network, in which a plurality of computers are connected to each other to exchange information. However, the Internet network is an example, and the spirit of the disclosure is not limited thereto.

Therefore, the communication network 500 may include one or more networks among networks, such as a personal area network (PAN), a local area network (LAN), a campus area network (CAN), a metropolitan area network (MAN), a wide area network (WAN), and a broad band network (BBN).

Also, the communication network 500 may include one or more of a network topology including a bus network, a start network, a ring network, a mesh network, a star-bus network, and a tree or hierarchical network, but is not limited thereto.

The server 300 may be implemented as a computer device or a plurality of computer devices, each providing commands, code, files, content, services, and the like to the patient terminal 200 and the medical team terminal 400 via the communication network 500.

For example, the server 300 may provide a file for installing an application to the patient terminal 200 and the medical team terminal 400, which are accessed via the communication network 500. The patient terminal 200 and the medical team terminal 400 may install the application by using the file provided from the server 300.

For example, the server 300 may provide an application file for measuring a bio-signal to the patient terminal 200, and may provide an application file for monitoring states of a plurality of patients to the medical team terminal 400.

Also, the patient terminal 200 and the medical team terminal 400 may receive a service or content provided by the server 300 by accessing the server 300 according to the control by an operating system (OS) and at least one program (for example, a browser or an installed application), which are included in the patient terminal 200 and the medical team terminal 400. For example, when the medical team terminal 400 requests bio-signal history data of a specific patient via the communication network 500, the server 300 may transmit the bio-signal history data of the patient to the medical team terminal 400, in response to the request. In this regard, the medical team terminal 400 may display the bio-signal history data according to the control by an application and/or the control by a web browser and provide the bio-signal history data to a medical team.

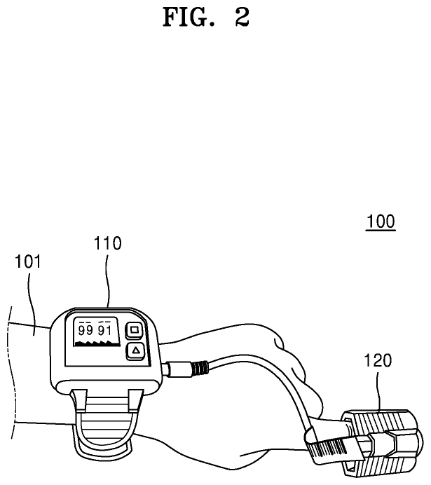

FIG. 2 is a diagram for explaining a configuration of the bio-signal measurement device 100 configured to measure a saturation level of oxygen in the body and a heart rate, according to an embodiment of the disclosure.

When the bio-signal measurement device 100 measures a saturation level of oxygen in the body and a heart rate of a patient 101, a main body unit 110 of the bio-signal measurement device 100 may be attached to a wrist of the patient 101, and a measurement unit 120 of the bio-signal measurement device 100 may be attached to a finger end of the patient 101, as shown in FIG. 2.

Also, the measurement unit 120 may measure at least one of the saturation level of oxygen in the body and the heart rate of the patient 101 according to the control by the main body unit 110 and transmit the at least one of at least one of the saturation level of oxygen in the body and the heart rate of the patient 101 to the main body unit 110. In this regard, the measurement unit 120 and the main body unit 110 may transmit/receive data and/or control commands via wired connection.

In addition, the configuration of the bio-signal measurement device 100 shown in FIG. 2 is an example, and the spirit of the disclosure is not limited thereto. Therefore, the bio-signal measurement device 100 may be formed as a single integrated main body unit (not shown) in which the main body unit 110 and the measurement unit 120 are integrated, and may be attached to other body parts of the patient 101.

In an embodiment, the main body unit 110 of the bio-signal measurement device 100 may include a general-purpose interface (not shown) for connection with various types of measurement units 120. For example, the main body unit 110 may include an interface (not shown) that is interchangeably connected with various measurement units, such as a measurement unit for measuring blood glucose and a measurement unit for measuring body temperature, in addition to the measurement unit 120 for measuring a saturation level of oxygen in the body and a heart rate.

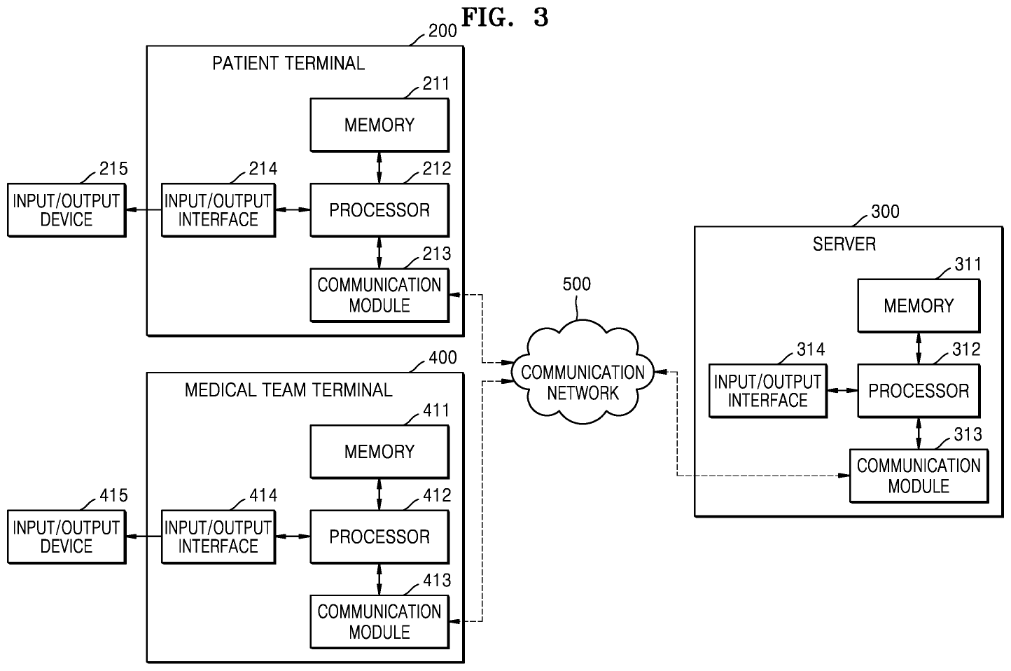

FIG. 3 is a block diagram for explaining internal configurations of the patient terminal 200, the medical team terminal 400, and the server 300, according to an embodiment of the disclosure.

The patient terminal 200, the medical team terminal 400, and the server 300 may include memories 211, 411, and 311, processors 212, 412, and 312, communication modules 213, 413, and 313, and input/output interfaces 214, 414, and 314.

The memories 211, 411, and 311 are computer-readable recording media, and may include random access memory (RAM), read only memory (ROM), and a permanent mass storage device, such as a disk drive. In this regard, the memories 211, 411, and 311 may store an OS and at least one piece of program code (for example, code for measuring and transmitting bio-signals, which is installed and driven in the patient terminal 200).

These software components may be loaded from a computer-readable recording medium distinct from the memories 211, 411, and 311 by using a drive mechanism. The discrete computer-readable recording medium may include a computer-readable recording medium, such as a floppy drive, a disk, a tape, a digital versatile disc (DVD)/compact disc (CD)-ROM drive, and a memory card.

In another embodiment, the software components may be loaded into the memories 211, 411, and 311 via the communication modules 213, 413, and 313 rather than the computer-readable recording medium. For example, at least one program may be loaded into the memories 211, 411, and 311, based on a program installed by files provided by a file distribution system (for example, the server 300) for distributing application installation files via the communication network 500.

The processors 212, 412, and 312 may be configured to perform basic arithmetic, logic, and input/output operations to process a command of a computer program. The command may be provided to the processors 212, 412, and 312 by the memories 211, 411, and 311 or the communication modules 213, 413, and 313. For example, the processors 212, 412, and 312 may be configured to execute a command received according to program code stored in a recording device, such as the memories 211, 411, and 311.

The communication modules 213, 413, and 313 may provide a function for the patient terminal 200, the medical team terminal 400, and the server 300 to communicate with each other via the communication network 500. For example, a request generated by the processors 212 and 412 of the patient terminal 200 and the medical team terminal 400 according to program code stored in a recording device, such as the memories 211 and 411, may be transmitted to the server 300 via the communication network 500, according to the control by the communication modules 213 and 413. Conversely, a control signal, a command, content, a file, or the like provided according to the control by the processor 312 of the server 300 may transmit through the communication module 313 and the communication network 500, and then may be received by the patient terminal 200 and the medical team terminal 400 via the communication modules 213 and 413, respectively.

Also, the communication modules 213, 413, and 313 may provide a function for communicating with other devices without involving the communication network 500. For example, the communication module 213 of the patient terminal 200 may provide a function for receiving bio-signal data of a patient from the bio-signal measurement device 100.

The input/output interfaces 214, 414, and 314 may be means for interfaces with input/output devices 215 and 415. In this regard, the input device may include, for example, a device, such as a keyboard or a mouse, and the output device may include a device, such as a display for displaying a bio-signal measurement result or the like.

As another example, the input/output interfaces 214, 414, and 314 may be means for an interface with a device in which input and output functions are integrated into one, such as a touchscreen.

Also, in another embodiments, the patient terminal 200, the medical team terminal 400, and the server 300 may include more components than those shown in FIG. 3. However, there is no need to clearly show most of the prior art components.

For example, the patient terminal 200 and the medical team terminal 400 may include at least some of the input/output devices 215 and 415, or may further include other components, such as a transceiver, a global positioning system (GPS) module, a camera, other sensors, and a database.

Hereinafter, a method, performed by the patient adverse reaction prediction system described in FIG. 1, of identifying a bio-signal of a patient in real time and based on the bio-signal, predicting an adverse event is described.

The bio-signal measurement device 100 according to an embodiment of the disclosure may measure a bio-signal of a patient at a first time interval, and may transmit the measured result to the patient terminal 200 at the first time interval. In this regard, the first time interval is a value that is preset by a patient and/or a medical team or set when the bio-signal measurement device 100 is manufactured, and may be, for example, 1 second or 0.5 seconds. For example, the bio-signal measurement device 100 may measure a bio-signal of a patient every second and transmit the measured bio-signal to the patient terminal 200 every second.

The patient terminal 200 according to an embodiment of the disclosure may collect the bio-signal measured at the first time interval from the bio-signal measurement device 100 and transmit the collected bio-signal to the server 300 at a second time interval. In this regard, as the first time interval, the second time interval is also a value that is preset or set by a patient and/or a medical team, and may be, for example, 1 second or 0.5 seconds. The second time interval may be the same as the first time interval or may be longer than the first time interval.

For example, when the bio-signal measurement device 100 measures a bio-signal of a patient every second and transmit the measured bio-signal to the patient terminal 200 every second, the patient terminal 200 may also transmit the bio-signal collected every second to the server 300.

In addition, according to the state of a resource and/or the state of a communication network, the patient terminal 200 may transmit a bio-signal collected at a time interval longer than the first time interval to the server 300. For example, the patient terminal 200 may transmit a bio-signal collected every 2 seconds or 5 seconds longer than the first time interval, which is 1 second, to the server 300. In this case, the collected bio-signal transmitted by the patient terminal 200 to the server 300 may be an accumulation of a bio-signal transmitted by the bio-signal measurement device 100 for the second time interval. In an embodiment, the patient terminal 200 may analyze bio-signals accumulated for the second time interval in a predetermined manner, and only when it is determined that a patient is abnormal as a result of the analysis, may transmit the collected bio-signals and/or the analysis result to the server 300. In this regard, the server 300 may omit some of an analysis process by referring to the analysis result of the patient terminal 200, or may take action on the patient based on the analysis result of the patient terminal 200.

As such, the disclosure may appropriately adjust the second time interval in relation to the first time interval in response to various events.

The server 300 according to an embodiment of the disclosure may receive the bio-signal from the patient terminal 200 at the second time interval, and based on the received bio-signal, may identify whether a predetermined action is necessary for the patient at the second time interval. Also, when it is determined that the predetermined action is necessary for the patient, the server 300 may transmit a message to the medical team terminal 400.

In this regard, the server 300 according to an embodiment of the disclosure may determine whether the predetermined action is necessary for the patient, considering a past bio-signal history of the patient. For example, when the server 300 receives a bio-signal of a patient from the patient terminal 200 at a first time point, the server 300 may determine whether a pattern of the bio-signal (for example, an oxygen saturation level pattern and a heart rate pattern) within a predetermined time period from the first time point (to the past) corresponds to a pre-stored adverse reaction pattern, and take appropriate action in response to a result of the determination.

Also, the server 300 according to an embodiment of the disclosure may further receive information about a current state of the patient from the patient terminal 200, and may determine whether the predetermined action is necessary for the patient by referring to the received state information. In this regard, the server 300 may pre-obtain and store an oxygen saturation level pattern and a heart rate pattern for each state for each patient.

For example, the server 300 may receive state information, such as exercising, sleeping, driving, and rehabilitating, from the patient terminal 200, and may determine whether a predetermined action is necessary for a patient by comparing an oxygen saturation level pattern and/or heart rate pattern corresponding to each piece of state information to a current oxygen saturation level pattern and/or current heart rate pattern.

As such, the server 300 according to an embodiment of the disclosure may generate and store a patient-specific profile including a state-specific oxygen saturation level pattern and/or heart rate pattern.

Accordingly, the disclosure may detect an adverse event in a patient in real time by monitoring a bio-signal of the patient in real time, and may more accurately detect an adverse event considering the specificity of individual patients.

In addition, when determining that the predetermined action is necessary for the patient, the server 300 according to an embodiment of the disclosure may request that the patient terminal 200 transmit a bio-signal to the server 300 at a second-first time interval (for example, 2 seconds) shorter than the second time interval (for example, 4 seconds).

Also, the server 300 may request via the patient terminal 200 that the bio-signal measurement device 100 measure a bio-signal of the patient at a first-first time interval (for example, 1 second) shorter than the first time interval (for example, 2 seconds) and transmit the bio-signal to the patient terminal 200.

Accordingly, the server 300 may receive the bio-signal from the patient terminal 200 at the second-first time interval (for example, 2 seconds), and may identify whether an additional action is necessary for the patient at the second-first time interval (for example, 2 seconds).

Also, the server 300 may collect the bio-signal received at the second-first time interval (for example, 2 seconds) and transmit a message to the medical team terminal 400 at a third time interval (for example, 10 seconds). In this regard, the third time interval may also be appropriately set by a user, such as a medical team.

As such, the disclosure may identify a real-time state of a patient from a remote place by reducing a time difference between an actual state of the patient and a state identified by a medical team when an adverse event occurs to the patient, and may more effectively utilize resources of the bio-signal measurement device 100 and the patient terminal 200 by transmitting a bio-signal according to a relatively long time interval when an adverse event does not occur.

When the patient terminal 200 according to an embodiment of the disclosure does not receive the bio-signal from the bio-signal measurement device 100 at the first time interval or does not transmit the collected bio-signal to the server 300 at the second time interval, the patient terminal 200 may generate an alarm in a preset manner.

For example, the patient terminal 200 may generate an alarm when the patient terminal 200 does not receive the bio-signal even after the first time interval has elapsed due to a lack of battery of the bio-signal measurement device 100 and/or a communication failure with the bio-signal measurement device 100.

Also, the patient terminal 200 may also generate an alarm even when the patient terminal 200 does not transmit the collected bio-signal to the server 300 due to poor connection with the communication network 500. A patient or another user who recognizes a failure event according to the alarm may take appropriate action to ensure that the bio-signal is transmitted smoothly, thereby ensuring real-time monitoring of bio-signals.

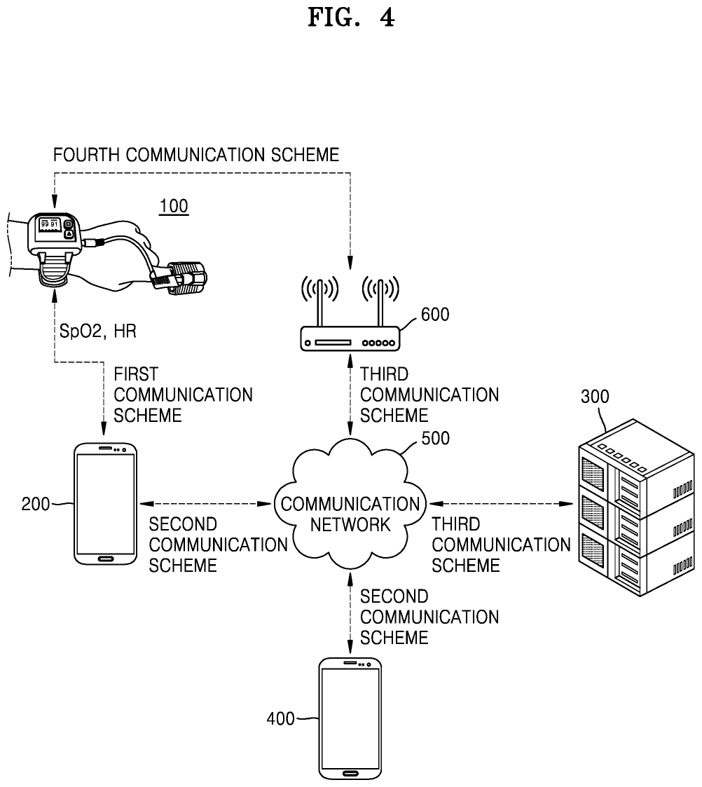

FIG. 4 is a diagram for explaining a communication scheme between each element of an adverse reaction prediction system, according to an embodiment of the disclosure. Hereinafter, a communication scheme between the bio-signal measurement device 100, the patient terminal 200, the server 300, and the medical team terminal 400 is described with reference to FIG. 4.

First, the bio-signal measurement device 100 and the patient terminal 200 according to an embodiment of the disclosure may be connected via a first communication scheme. Both of the bio-signal measurement device 100 and the patient terminal 200 may be attached to the body of a patient or located near the patient, and thus, may be connected via a near-field communication scheme, such as a Bluetooth scheme, a worldwide interoperability for microwave access (WiMAX) scheme, a wireless fidelity (Wi-Fi) scheme, a ZigBee scheme, a wireless body area network (WBAN) scheme, and a medical body area network (MBAN).

For example, the bio-signal measurement device 100 and the patient terminal 200 may be paired by a Bluetooth scheme to transmit/receive data. A time interval for transmitting/receiving data in the Bluetooth scheme may be the first time interval as described above.

In addition, the patient terminal 200 and the medical team terminal 400 may be connected to the communication network 500 via a second communication scheme. In this regard, the second communication scheme may be a scheme generally used for communication of a mobile communication terminal, such as an LTE scheme, an LTE advanced scheme, an EV-DO scheme, a Wibro scheme, an HSPDA scheme, a CDMA2000 scheme, a GSM scheme, and a WCDMA scheme.

Although not shown in FIG. 4, the second communication scheme may connect the patient terminal 200 and the medical team terminal 400 to the communication network 500 via at least one base station and/or a relay device.

For example, the patient terminal 200 and the medical team terminal 400 are portable phones, and may be connected to an Internet network via an LTE scheme. A time interval for transmitting/receiving data to/from the server 300 in the LTE scheme may be the second time interval (in the case of the patient terminal 200) and the third time interval (in the case of the medical team terminal 400) as described above.

The server 300 may be connected to the communication network 500 via a third communication scheme. In this regard, the third communication scheme may be a wired connection scheme, such as a DSL scheme, an xDSL scheme, an HFC scheme, an FTTO scheme, an FTTC scheme, an FTTH scheme, an LMDS scheme, and an MMDS scheme.

Although not shown in FIG. 4, the third communication scheme may connect the server 300 to the communication network 500 via at least one network router, an internet data center (IDC), and/or a relay device.

As such, the disclosure may enable the bio-signal measurement device 100 to be miniaturized by allowing communication, which requires a relatively large amount of resources, to be performed by the patient terminal 200, and accordingly, increase the ease of attachment of the bio-signal measurement device 100 to the body of a patient, and reduce manufacturing cost of the bio-signal measurement device 100.

According to an embodiment of the disclosure, the bio-signal measurement device 100 may be directly connected to the communication network 500 to transmit a bio-signal. In this case, the bio-signal measurement device 100 may be connected to the communication network 500 by a device that mediates the communication network 500 and the bio-signal measurement device 100, such as a wireless router 600.

When the bio-signal measurement device 100 according to an embodiment of the disclosure is not capable of transmitting a bio-signal to the patient terminal 200 via the first communication scheme, the bio-signal measurement device 100 may be directly connected to the communication network 500 via a fourth communication scheme temporarily, and then transmit the bio-signal to the patient terminal 200 and/or the server 300 via the communication network 500.

In this regard, data transmission/reception according to the fourth communication scheme consumes more power and requires more resources than data transmission/reception according to the first communication scheme, wherein the fourth communication scheme may be one of a Wi-Fi scheme, a WBAN scheme, and an MBAN scheme, and the first communication scheme may be a Bluetooth scheme.

The patient terminal 200 may transmit the bio-signal received from the bio-signal measurement device 100 via the communication network 500 to the server 300 via the communication network 500. When the bio-signal measurement device 100 directly transmits the bio-signal to the server 300 via the communication network 500, at least some of the described process may be omitted, and the server 300 may store identification information of a plurality of bio-signal measurement devices 100 by respectively mapping the identification information with patients.

For example, when a patient leaves his/her portable phone (that is, the patient terminal 200) and moves to another place or due to the battery of the portable phone getting low, a bio-signal measured by the bio-signal measurement device 100 may not be transmitted to the server 300.

In this case, there may be a blank period in monitoring of a bio-signal until the patient retrieves the portable phone or recharges the portable phone, and an adverse event in the patient during the blank period may not be identified.

Therefore, when the bio-signal measurement device 100 according to an embodiment of the disclosure is not capable of transmitting the bio-signal to the patient terminal 200 via the first communication scheme, the bio-signal measurement device 100 may be connected to the communication network 500 via the fourth communication scheme temporarily, and may directly transmit the bio-signal to the server 300 or transmit the bio-signal to the server 300 via the patient terminal 200.

Accordingly, the disclosure may identify an adverse event in a patient without a blank period in spite of various failure events.

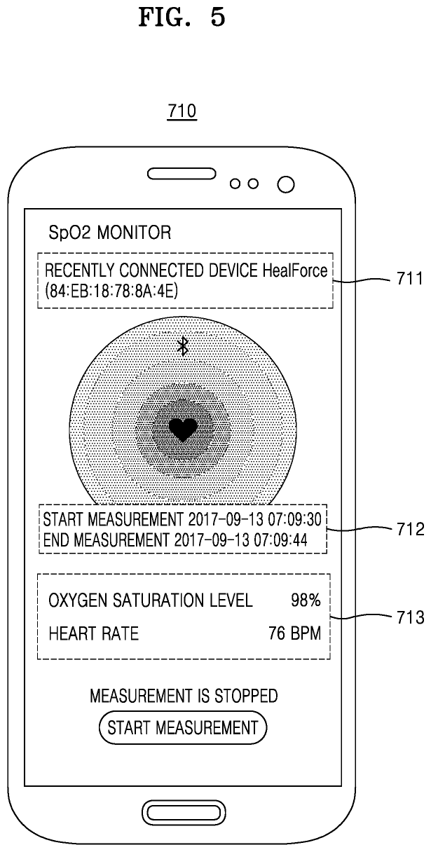

FIG. 5 is an example of a measurement result screen 710 displayed on the patient terminal 200, according to an embodiment of the disclosure.

Referring to FIG. 5, the measurement result screen 710 may include an area 711 for displaying identification information of the bio-signal measurement device 100, an area 712 for displaying additional information about measurements, and an area 713 for displaying a measurement result.

Via the measurement result screen 710, a patient may not only identify his/her current state, but also check a measurement result and measurement-related information, and may check a connection state of the bio-signal measurement device 100.

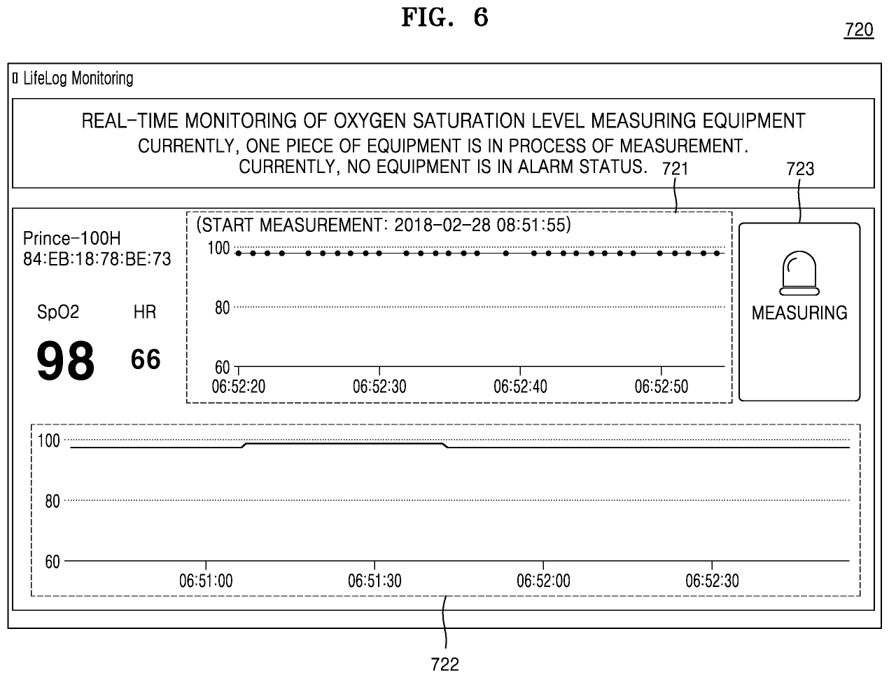

FIG. 6 is an example of a monitoring screen 720 for a specific patient, which is displayed on the server 300, according to an embodiment of the disclosure.

Referring to FIG. 6, the monitoring screen 720 may include an area 721 for displaying an oxygen saturation level graph over time for a specific patient, an area 722 for displaying a heart rate graph over time, and an area 723 for displaying a result of determining a current state.

A medical team may predict an adverse reaction of a patient by comprehensively referring to an oxygen saturation level and/or heart rate over time for the patient and a state determination result via the monitoring screen 720 of the server 300, and may take appropriate action (for example, a hospital visit request) according to a result of the prediction.

In addition, a monitoring result for only one patient is shown in FIG. 6, but is an example, and monitoring results for a plurality of patients may be displayed on one screen.

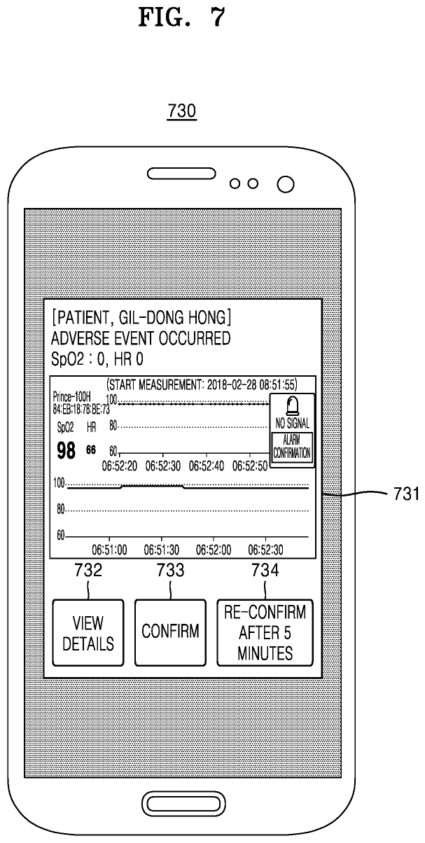

FIG. 7 is an example of a message reception screen 730 displayed on the medical team terminal 400 in the event of an adverse event in a specific patient.

As described above, when it is determined that a predetermined action is necessary for a patient, the server 300 may transmit a message to the medical team terminal 400. Accordingly, the medical team terminal 400 may receive the message as in the message reception screen 730.

In this regard, the message reception screen 730 may include a pop-up window 731 for displaying patient identification information, oxygen saturation level information, heart rate information, and accordingly various options 732, 733, and 734.

In addition, a medical team may select the option to view details 732 to check more detailed information of a patient, and may transmit a hospital visit request according to a result of the check.

When the medical team selects an option to view details 732, the medical team terminal 400 may request detailed information from the server 300 and display the detailed information by receiving the detailed information from the server 300.

When no special action is required for a patient or an action has been completed, the medical team may select an option to confirm 733 to allow the pop-up window 731 to disappear.

In addition, when it is determined that the patient's progress needs to be monitored a little longer, the medical team may select an option to re-confirm after 5 minutes 734 to re-check a state of the patient after 5 minutes. In this case, after counting a preset time, the medical team terminal 400 according to an embodiment of the disclosure may request for updated patient information from the server 300, and re-display the updated patient information by receiving the updated patient information from the server 300.

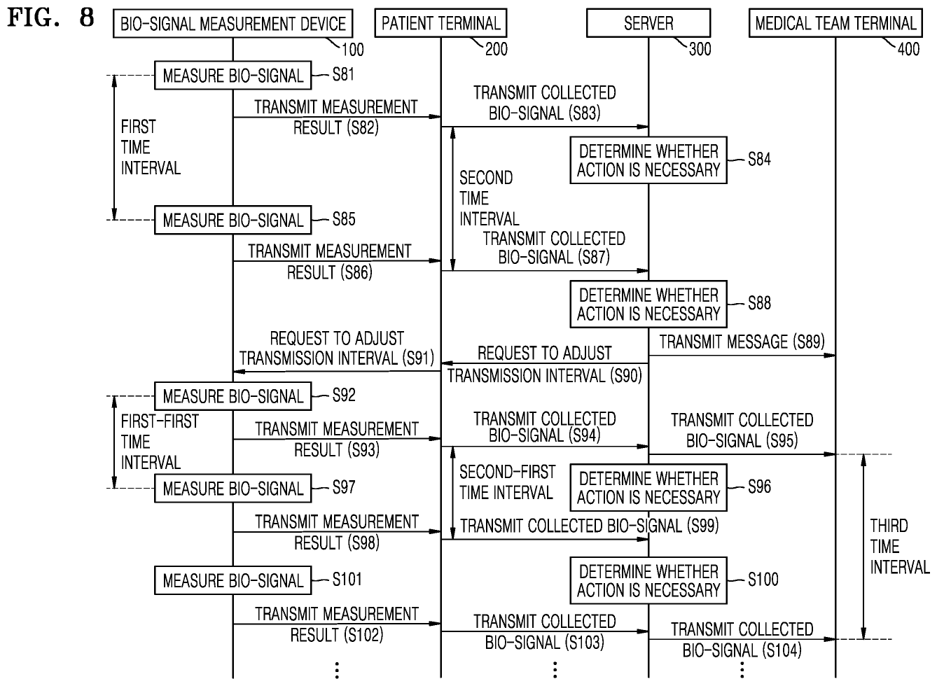

FIG. 8 is a flowchart for explaining a method, performed by a patient adverse reaction prediction system, of identifying a bio-signal of a patient in real time, and a method of predicting an adverse event in a patient based thereon. Hereinafter, descriptions that are already given with reference to FIGS. 1 to 7 are omitted.

The bio-signal measurement device 100 according to an embodiment of the disclosure may measure a bio-signal of a patient (operation S81). Also, the bio-signal measurement device 100 may transmit a measurement result to the patient terminal 200 (operation S82).

As described above, the bio-signal measurement device 100 may repeatedly perform operation S81 and operation S82 at a first time interval. For example, the bio-signal measurement device 100 may repeatedly measure a bio-signal of the patient after the first time interval from a time point at which operation S81 is performed (operation S85). Also, the bio-signal measurement device 100 may repeatedly transmit a measurement result to the patient terminal 200 after the first time interval from a time point at which operation S82 is performed (operation S86).

In this regard, the first time interval is a value that is preset by a patient and/or a medical team or set when the bio-signal measurement device 100 is manufactured, and may be, for example, 1 second or 0.5 seconds. For example, the bio-signal measurement device 100 may measure a bio-signal of a patient every second and transmit the measured bio-signal to the patient terminal 200 every second.

The patient terminal 200 according to an embodiment of the disclosure may collect the bio-signal measured at the first time interval from the bio-signal measurement device 100 and transmit the collected bio-signal to the server 300 at a second time interval (operation S83 and operation S87). In this regard, as the first time interval, the second time interval is also a value that is preset by a patient and/or a medical team or set, and may be, for example, 1 second or 0.5 seconds. The second time interval may be the same as the first time interval or may be longer than the first time interval.

For example, when the bio-signal measurement device 100 measures a bio-signal of a patient every second and transmit the measured bio-signal to the patient terminal 200 every second, the patient terminal 200 may also transmit the bio-signal collected every second to the server 300.

In addition, according to the state of a resource and/or the state of a communication network, the patient terminal 200 may transmit a bio-signal collected every 2 seconds or 5 seconds longer than the first time interval to the server 300. In this case, the collected bio-signal transmitted by the patient terminal 200 to the server 300 may be an accumulation of a bio-signal transmitted by the bio-signal measurement device 100 for the second time interval. As such, the disclosure may appropriately adjust the second time interval in response to various events.

The server 300 according to an embodiment of the disclosure may identify whether a predetermined action is necessary for the patient at the second time interval, based on the received bio-signal (operation S84 and operation S88).

In this regard, the server 300 according to an embodiment of the disclosure may determine whether the predetermined action is necessary for the patient, considering a past bio-signal history of the patient. For example, when the server 300 receives a bio-signal of a patient from the patient terminal 200 at a first time point, the server 300 may determine whether a pattern of the bio-signal (for example, an oxygen saturation level pattern and a heart rate pattern) within a predetermined time period from the first time point (to the past) corresponds to a pre-stored adverse reaction pattern, and take appropriate action in response to a result of the determination.

Accordingly, the disclosure may detect an adverse event in a patient in real time by monitoring a bio-signal of the patient in real time, and may more accurately detect an adverse event by detecting the adverse event considering the specificity of individual patients.

When it is determined that the predetermined action is necessary for the patient, the server 300 according to an embodiment of the disclosure may transmit a message to the medical team terminal 400 (operation S89).

When determining that the predetermined action is necessary for the patient, the server 300 according to an embodiment of the disclosure may request that the patient terminal 200 transmit a bio-signal to the server 300 at a second-first time interval (for example, 2 seconds) shorter than the second time interval (for example, 4 seconds) (operation S90). Also, the server 300 may request via the patient terminal 200 that the bio-signal measurement device 100 measure a bio-signal of the patient at a first-first time interval (for example, 1 second) shorter than the first time interval (for example, 2 seconds) and transmit the bio-signal to the patient terminal 200 (operation S91).

Accordingly, the bio-signal measurement device 100 according to an embodiment of the disclosure may repeatedly measure a bio-signal according to the first-first time interval (operation S92, operation S97, and operation S101), and may repeatedly transmit a measurement result to the patient terminal 200 (operation S93, operation S98, and operation S102).

Also, the patient terminal 200 may also collect the bio-signal measured at the first-first time interval from the bio-signal measurement device 100 and repeatedly transmit the collected bio-signal to the server 300 at the second-first time interval (operation S94, operation S99, and operation S103).

Furthermore, the server 300 according to an embodiment of the disclosure may receive the bio-signal from the patient terminal 200 at the second-first time interval, and based on the received bio-signal, may identify whether a predetermined action is necessary for the patient at the second-first time interval (operation S96 and operation S100).

As such, the disclosure may identify a real-time state of a patient from a remote place by reducing a time difference between an actual state of the patient and a state identified by a medical team when an adverse event occurs to the patient, and may more effectively utilize resources of the bio-signal measurement device 100 and the patient terminal 200 by transmitting a bio-signal according to a relatively long time interval when an adverse event does not occur.

In addition, when it is determined that the predetermined action is necessary for the patient in operation S88, the server 300 may then transmit biometric information collected from the patient terminal 200 to the medical team terminal 400 at a third time interval (operation S95 and operation S104).

Therefore, the disclosure may allow a medical team to more conveniently check various pieces of information about a patient without physical access to the server 300 or the like, and to more easily identify the progress by providing various pieces of information about a patient at a predetermined third time interval.

The device described above may be implemented as a hardware component, a software component, and/or a combination of the hardware component and the software component. For example, the devices and components described in the embodiments may be implemented by using one or more general-purpose computers or special-purpose computers, such as a processor, a controller, an arithmetic logic unit (ALU), a digital signal processor, a field programmable gate array (FPGA), a programmable logic unit (PLU), a microprocessor, or any other device capable of executing and responding to commands. A processing device may execute an OS and one or more software applications executed on the OS. Also, the processing device may access, store, manipulate, process, and create data in response to execution of software. For convenience of understanding, the processing device is described as singular, but those of ordinary skill in the art will appreciate that the processing device may include a plurality of processing elements and/or a plurality types of processing elements. For example, the processing device may include a plurality of processors or one processor and one controller. Also, different processing configurations are possible, such as a parallel processor.

The software may include a computer program, a piece of code, a command, or a combination of one or more thereof, and may configure the processing device to operate as desired or independently or collectively command the processing device. The software and data may be embodied permanently or temporarily in any type of machine, component, physical or virtual equipment, computer storage medium or device, or in a propagated signal wave in order to be interpreted by the processing device or to provide commands or data to the processing device. The software is distributed over network-coupled computer systems so that the software may be stored and executed in a distributed manner. The software and data may be stored in one or more computer-readable recording mediums.

The methods according to the embodiments may be implemented in the form of program commands that may be executed via various computer means, and may be recorded on a computer-readable medium. The computer-readable medium may include program instructions, data files, data structures, and the like alone, or may include a combination thereof. The program commands recorded on the medium may be specially designed and configured for the embodiment, or may be known and available to those of ordinary skill in the art of computer software. Examples of the computer-readable medium include magnetic media such as hard disks, floppy disks, and magnetic tape, optical media such as CD-ROMs and DVDs, magneto-optical media such as floptical discs, and hardware devices that are specially configured to store and perform program commands, such as ROM, RAM, and flash memory. Examples of the program command may include not only machine language code produced by a compiler but also high-level language code that is executable by a computer by using an interpreter or the like. The hardware devices may be configured to operate as one or more software modules in order to perform the operations of the embodiments, or vice versa.

As described above, although the examples have been described by a limited embodiment and drawings, those of skill in the art may make various modifications and variations from the above description. For example, even in a case where the described techniques are performed in a different order than the described method, and/or the components of the described system, structure, device, circuit, etc. are combined in a different form from the described method or are replaced or substituted by equivalents, appropriate results may be achieved.

Therefore, other implementations, other embodiments, and equivalents to the claims are also within the scope of the following claims.

1. A system for predicting an adverse reaction of a patient, based on a real-time bio-signal of the patient, the system comprising:
a bio-signal measurement device configured to measure a bio-signal of the patient at a first time interval and transmit a measured result to a patient terminal at the first time interval;
the patient terminal configured to collect the bio-signal measured at the first time interval from the bio-signal measurement device and transmit the collected bio-signal to a server at a second time interval;
the server configured to receive the bio-signal from the patient terminal at the second time interval, based on the received bio-signal, identify whether a predetermined action is necessary for the patient at the second time interval, and when the predetermined action is necessary, transmit a message to a medical team terminal; and
the medical team terminal configured to receive the message from the server when the predetermined action is necessary.

2. The system of claim 1, wherein the bio-signal of the patient comprises at least one of a saturation level of oxygen in a body of the patient and a heart rate of the patient.

3. The system of claim 2, wherein the server is configured to receive the bio-signal of the patient from the patient terminal at a first time point, and
when at least one pattern of an oxygen saturation level pattern and a heart rate pattern within a predetermined time period from the first time point corresponds to a pre-stored adverse reaction pattern, determine that the predetermined action is necessary for the patient, and transmit a message including at least one of the oxygen saturation level pattern and the heart rate pattern to the medical team terminal.

4. The system of claim 3, wherein the server is configured to, when it is determined that the predetermined action is necessary for the patient, request the patient terminal to transmit the bio-signal to the server at a second-first time interval that is shorter than the second time interval,
request that the bio-signal measurement device measure the bio-signal of the patient at a first-first time interval that is shorter than the first time interval via the patient terminal and transmit the measured bio-signal to the patient terminal, and
receive the bio-signal from the patient terminal at the second-first time interval and identify whether an additional action is necessary for the patient at the second-first time interval.

5. The system of claim 4, wherein the server is configured to, when it is determined that the predetermined action is necessary for the patient, collect the bio-signal received at the second-first time interval and transmit the message to the medical team terminal at a third time interval.
